(19) United States
(12) Patent Application Publication
(10) Pub. No.: US 2025/0135204 A1
(43) Pub. Date: May 1, 2025

(54) SYSTEM AND METHOD FOR REMOVING STIMULATION ARTIFACT IN NEUROMODULATION SYSTEMS

(71) Applicant: THE JOHNS HOPKINS UNIVERSITY, Baltimore, MD (US)

(72) Inventors: William Stanley ANDERSON, Towson, MD (US); Yousef SALIMPOUR, Baltimore, MD (US); Kelly MILLS, Silver Spring, MD (US)

(21) Appl. No.: 18/690,782

(22) PCT Filed: Sep. 14, 2022
(86) PCT No.: PCT/US2022/043448

§ 371 Date: Mar. 11, 2024

Related U.S. Application Data
(60) Provisional application No. 63/245,358, filed on Sep. 17, 2021.

(51) Int. Cl. A61N 1/36

(52) U.S. Cl. CPC A61N 1/36139; A61N 1/36171

(57) ABSTRACT

A method, computer system, and a non-transitory computer readable medium are disclosed that performs instructions including receiving, from one or more electrodes, information identifying brain activity with a stimulus artifact signal for a first time period; predicting, based on the information identifying the brain activity for the first time period, predicted brain activity without the stimulus artifact signal for a second time period that is to occur after the first time period; inserting the predicted brain activity into the information for a second time period; determining, based on the predicted brain activity for the second time period, a brain stimulus for the second time period; and causing the brain stimulus to be applied the second time period.

### CROSS-REFERENCE TO RELATED APPLICATIONS

This application is the national stage entry of International Patent Application No. PCT/US2022/043448, filed on Sep. 14, 2022, and published as WO 2023/043784 A1 on Mar. 23, 2023, which claims the benefit of U.S. Provisional Patent Application Ser. No. 63/245,358, filed on Sep. 17, 2021, the disclosures of which are hereby incorporated by reference in their entireties.

### FIELD

This application is directed to neuromodulation systems, and in particular, to systems and methods for removing stimulation artifacts in neuromodulation systems.

### BACKGROUND

Brain stimulation may be used to alter, enhance, and/or improve brain function in neurological disorders, such as epilepsy, movement disorders, and/or the like. A control device may stimulate a brain of a patient by causing electrical stimulation pulses to be applied to the brain using a set of electrodes. The control device may trigger the electrodes to provide a constant, repeating set of electrical stimulation pulses, which may cause brain function to be altered, enhanced, and/or improved. The set of electrodes may be disposed on the patient's brain surface, or within the brain substance and may be connected to the control device via a set of wires.

Neuromodulation is a rapidly expanding area of translational neuroscience that involves stimulation, excitation, inhibition, and alteration of activity in the nervous system using electrical, electromagnetic, chemical, and even mechanical stimuli. The use of electrophysiological recordings has widely increased in modern neuromodulation technologies, specifically in closed-loop neuromodulation applications to increase the efficacy of neuromodulation based clinical treatments. Currently, a variety of neuromodulation techniques are being utilized for the treatment of neurological disorders such as Parkinson's disease and other movement disorders, chronic pain, psychiatric disorders, epilepsy, and many others. Electrical neuromodulation represents electrical or electromechanical stimulation of the nervous system in various structures such as the brain, spinal cord, and peripheral nerves.

The use of electrophysiological features as biofeedback in neuromodulation for adjusting the applied therapy characteristics is increasingly being used. In order to have an effective neuromodulation treatment, the system must be able to record and stimulate simultaneously during regular neural activities. However, persistent stimulation artifacts that distort recorded signals represent a severe challenge and so an effective artifact reduction technique is needed.

### SUMMARY

According to examples of the present disclosure, a method is disclosed that comprises receiving, from one or more electrodes, information identifying brain activity with a stimulus artifact signal for a first time period; predicting, based on the information identifying the brain activity for the first time period, predicted brain activity without the stimulus artifact signal for a second time period that is to occur after the first time period; inserting the predicted brain activity into the information for a second time period; determining, based on the predicted brain activity for the second time period, a brain stimulus for the second time period; and causing the brain stimulus to be applied the second time period.

Various additional features can be included in the method including one or more of the following features. The brain stimulus is associated with a frequency and a phase determined based on the predicted brain activity for the second time period. The method further comprises determining the stimulus artifact signal during the first time period associated with a prior brain stimulus; and determining an artifact-removed brain activity for the first time period based on the artifact. The predicting the brain activity for the second time period further comprises predicting the brain activity for the second time period based on the artifact-removed brain activity. The brain stimulus is caused to occur during a period of rhythmic brain activity. The predicting the brain activity for the second time period further comprises predicting the brain activity using a parametric spectral estimation technique for modeling band limited oscillations.

According to examples of the present disclosure, a device is disclosed that includes one or more memories; and one or more processors communicatively coupled to the one or more memories, configured to: receiving, from one or more electrodes, information identifying brain activity with a stimulus artifact signal for a first time period; predicting, based on the information identifying the brain activity for the first time period, predicted brain activity without the stimulus artifact signal for a second time period that is to occur after the first time period; inserting the predicted brain activity into the information for a second time period; determining, based on the predicted brain activity for the second time period, a brain stimulus for the second time period; and causing the brain stimulus to be applied the second time period.

Various additional features can be included in the device including one or more of the following features. The brain stimulus is associated with a frequency and a phase determined based on the predicted brain activity for the second time period. The one or more processors communicatively coupled to the one or more memories are further configured to determine the stimulus artifact signal during the first time period associated with a prior brain stimulus; and determine an artifact-removed brain activity for the first time period based on the artifact. The predicting the brain activity for the second time period further comprises: predicting the brain activity for the second time period based on the artifact-removed brain activity. The brain stimulus is caused to occur during a period of rhythmic brain activity. The predicting the brain activity for the second time period further comprises predicting the brain activity using a parametric spectral estimation technique for modeling band limited oscillations.

According to examples of the present disclosure, a non-transitory computer readable medium is disclosed that comprises instructions that when executed by a hardware processor cause the hardware processor perform a method, comprising: receiving, from one or more electrodes, information identifying brain activity with a stimulus artifact signal for a first time period; predicting, based on the information identifying the brain activity for the first time period, predicted brain activity without the stimulus artifact signal for a second time period that is to occur after the first time period; inserting the predicted brain activity into the information for a second time period; determining, based on the predicted brain activity for the second time period, a brain stimulus for the second time period; and causing the brain stimulus to be applied the second time period.

Various additional features can be included in the non-transitory computer readable medium including one or more of the following features. The brain stimulus is associated with a frequency and a phase determined based on the predicted brain activity for the second time period. The non-transitory computer readable medium further comprises determining the stimulus artifact signal during the first time period associated with a prior brain stimulus; and determining an artifact-removed brain activity for the first time period based on the artifact. The predicting the brain activity for the second time period further comprises: predicting the brain activity for the second time period based on the artifact-removed brain activity. The brain stimulus is caused to occur during a period of rhythmic brain activity. The predicting the brain activity for the second time period further comprises: predicting the brain activity using a parametric spectral estimation technique for modeling band limited oscillations.

### BRIEF DESCRIPTION OF THE DRAWINGS

The accompanying drawings, which are incorporated in and constitute a part of this specification, illustrate embodiments of the present teachings and together with the description, serve to explain the principles of the present teachings. In the figures:

FIG. 1 shows a system diagram according to examples of the present disclosure;

FIG. 2 shows an example environment in which systems and/or methods described herein may be implemented;

FIG. 3 shows a first example of a recording/prediction diagram according to examples of the present disclosure;

FIG. 4 shows a second example of a recording/prediction diagram according to examples of the present disclosure;

FIG. 5 shows a third example of a recording/prediction diagram according to examples of the present disclosure;

FIG. 6 shows a fourth example of a recording/prediction diagram according to examples of the present disclosure;

FIG. 7 shows a recorded signal with a modeled predicted signal portion added that has attributes of prediction, filtering, and smoothing according to examples of the present disclosure;

FIG. 8 shows a recording/prediction system diagram according to examples of the present disclosure;

FIG. 9 shows a surface electrode and depth electrode placement according to examples of the present disclosure;

FIG. 10 shows a cortical multichannel recording example during subcortical stimulation according to examples of the present disclosure: The performance of the artifact removal algorithm on an estimation of electrophysiological recording across all contacts in the strip electrode is shown here. The row data are recorded during motor cortex phase-dependent stimulation;

FIG. 11 shows an example of concurrent cortical stimulation and recording according to examples of the present disclosure: The performance of the artifact removal algorithm is presented with an estimation of electrophysiological recording during motor cortex recording and stimulation;

FIG. 12 shows an example of concurrent subcortical stimulation and cortical recording according to examples of the present disclosure: The performance of the artifact removal algorithm is presented with an estimation of electrophysiological recording during subthalamic nucleus stimulation and motor cortex recording.

FIG. 13 shows a method for artifact removal for neuromodulation systems according to examples of the present disclosure; and

FIG. 14 illustrates a schematic view of a computing system according to examples of the present disclosure.

### DETAILED DESCRIPTION

Reference will now be made in detail to embodiments, examples of which are illustrated in the accompanying drawings and figures. In the following detailed description, numerous specific details are set forth in order to provide a thorough understanding of the invention. However, it will be apparent to one of ordinary skill in the art that the invention may be practiced without these specific details. In other instances, well-known methods, procedures, components, circuits and networks have not been described in detail so as not to unnecessarily obscure aspects of the embodiments.

Electrical pulse-based brain stimulation may be performed using a fixed set of electrical pulses applied to a brain of a patient for a period of time. This technique may result in alteration, improvement, enhancement, and/or the like to brain function of the brain of the patient. However, using a constant set of electrical pulses may be inefficient, resulting in wasted energy resources, which may hinder miniaturization of brain stimulation devices. Moreover, using a fixed set of electrical pulses may result in relatively poor clinical outcomes. For example, using constant stimulation may result in lower thresholds for stimulation-associated side effects (e.g., as a result of activating or inhibiting structures proximate to locations at which the constant stimulation is applied).

Some implementations described herein use phase-dependent neuromodulation with stimulus artifact reduction or removal to reduce a utilization of energy resource, to improve a likelihood of positive patient outcomes from electrical pulse-based brain stimulation by modulating the cross-frequency coupling in the cortical structure, and/or the like. For example, a device may measure brain activity, predict future brain activity, reduces and/or removes stimulus artifacts, dynamically identify a stimulus pulse to control the predicted future brain activity, and cause the stimulus pulse to be applied to correct an issue with the predicted future brain activity. Moreover, based on reducing a utilization of energy resources, some implementations described herein enable miniaturization and/or implantability of a device to perform phase-dependent, stimulus artifact reduction or removal neuromodulation. This technique may be applicable in treatment relating to Parkinson's disease, theta rhythm issues relating to memory, Schizophrenia, Alzheimer's disease, and/or the like. The systems/methods described herein have the full flexibility to adapt to a variety of neuromodulation systems with the potential to combine with typical neuromodulation techniques including Transcranial Electrical Stimulation (TEs), Transcranial Magnetic Stimulation (TMS), Deep Brain Stimulation (DBS), Direct Cortical Stimulation (DCS), and Ultrasound therapies (US).

According to examples of the present disclosure, an adaptive artifact removal system for removing brain stimulation artifacts from the recording sites of a target brain structure used for recording or “sensing” is disclosed. For each recording channel of interest, we add two parallel blocks for a modeling algorithm, the first one for adaptive parametric modeling of the electrophysiology signal, and the second for predicting the signal in any time interval of interest using the parametric model. To allow for simultaneous stimulation and recording, the optimized parametric model is utilized for predicting the recorded signal during stimulation events. The predicted signal is substituted for the stimulus artifact during active stimulation. This removes the transient stimulation artifact and provides accurate electrophysiological signal detection even during stimulation. Since the stimulation events are relatively narrow, the duration of the artifact is a limited interval during which the output of the model has a low prediction error with no discontinuity occurring between the recorded signal versus model output. This method can be fully implemented on a system-on-chip (SoC) technology and easily added to existing neuromodulation devices. It also could be used for both offline and online stimulus artifact removal. In conclusion, our adaptive, model-based, and signal prediction-based stimulus artifact removal system has the potential for use in a wide range of brain stimulation methods including closed-loop neuromodulation systems.

Examples of this disclosure aim to reduce the stimulus artifact present on recording channels also subject to stimulation pulses. This is a challenge in almost any type of closed-loop neuromodulation system. Examples of this disclosure can be adopted for existing neuromodulation systems by adding a field programmable gate array or similar system on a chip design for running the necessary signal modeling.

Stimulus-induced artifacts distort the electrophysiological signal, alter feature detection, and significantly change parameter estimation. Stimulus artifact removal can increase the accuracy of brain recordings, and provide a more representative view of actual cortical network behavior. A. Here, an example of stimulus artifact removal utilizing the predicted signal from a predictive model is illustrated. The start time is synchronized with the stimulation trigger, and the stop time is estimated based on the stimulus artifact duration. B. To eliminate the stimulus artifact from the recordings, an optimized computational model was utilized for predicting the recorded signal during stimulation events. The predicted signal was substituted for the stimulus artifact during active stimulation x(t)=0 during the actual stimulation artifact, so that s(t)=x̅(t).

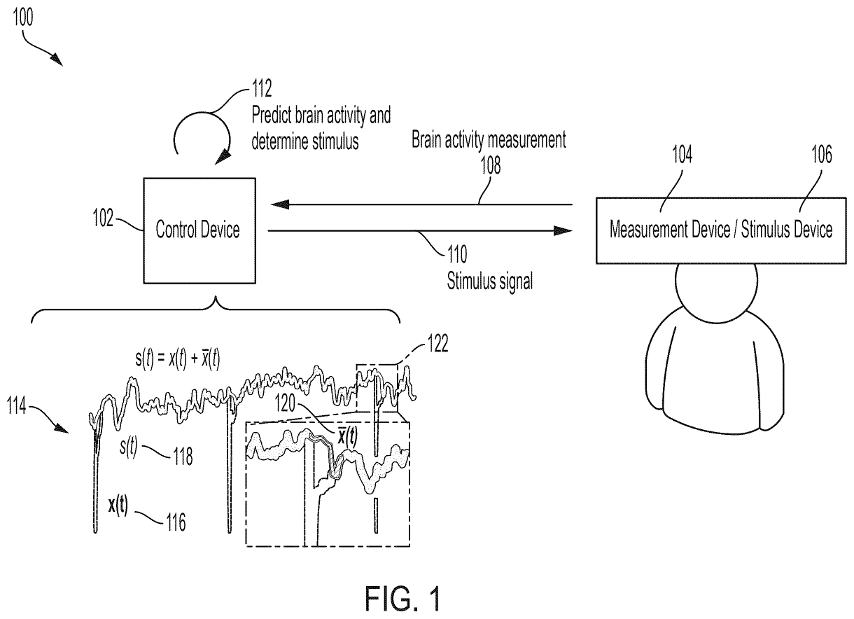

FIG. 1 shows a system diagram according to examples of the present disclosure. As shown in FIG. 1, example implementation 100 may include control device 102 that communicates with measurement device 104 and/or stimulus device 106. In some implementations, control device 102 or another device may wirelessly power measurement device 104 and/or stimulus device 106 via a wireless power transmission functionality.

As further shown in FIG. 1, and by reference number 108, control device 102 may receive information identifying a brain activity measurement from measurement device 104. For example, control device 102 may receive information identifying a phase, amplitude, frequency, and/or the like of brain activity of a brain of a patient. In some implementations, control device 102 may receive information identifying brain activity for a particular time interval. In some implementations, control device 102 may determine whether a threshold level of brain activity is detected for the particular time interval. For example, control device 102 may determine that a threshold level of beta activity (e.g., activity in a range of 13 Hertz (Hz) to 30 Hz) is detected. Additionally, or alternatively, control device 102 may determine that a threshold level of phase-amplitude coupling is detected. In this case, control device 102 may use a rolling dynamic phase amplitude coupling (PAC) estimation technique to determine the phase amplitude coupling in intervals of less than or equal to 1 second, 500 milliseconds, and/or the like. In this way, control device 102 may avoid performing complex calculations to predict brain activity when the brain activity is below a threshold for which corrective stimuli are to be applied, thereby reducing processor utilization, improving battery life, improving a lifespan of example implementation 100, and/or the like.

As further shown in FIG. 1, and by reference number 112, control device 102 may predict brain activity and determine a stimulus to apply to the brain of the patient. For example, and as shown by reference number 114, based on information identifying brain activity for a first time period, control device 102 may predict brain activity for a second time period occurring after the first time period. Reference number 114 is an example of a simulated recorded brainwave signal from a motor cortex of a patient, which comprises a first segment 116 representative of a stimulus signal and denoted by x(t), a second segment 118 representative of a recorded brainwave signal and denoted by s(t), and a third segment 120 representative by a modeled predictive brainwave signal with stimulus artifact removed and denoted by x(t), which are related by the following expression:

s(t)=x(t)+x̅(t)

where the portion of third segment 10 shown in box 112 in greater detail. In this case, control device 102 may determine a set of electrical pulses to correspond to a predicted period of rhythmic brain activity. For example, control device 102 may determine the set of electric pulses based on signal predictive modeling of rhythmic activity with forward-prediction to time the set of electric pulses in accordance with the rhythmic activity. Some examples may include using auto-regressive modeling, generalized linear modeling, machine learning-based modeling, and/or the like. In this way, control device 102 enables electrical pulse-based brain stimulation using reduced power and with improved efficacy relative to a constant set of brain rhythmic activities.

Control device 102 removes one or more artifacts from measured brain activity when determining predicted brain activity. For example, control device 102 may identify one or more artifacts in brain activity during a first time period corresponding to one or more electrical pulses provided during the first time period, and may remove the one or more artifacts in the brain activity to determine baseline brain activity without the one or more electrical pulses. In some implementations, control device 102 may predict the artifacts using signal predictive modeling to interpolate brain activity during periods when artifacts occur as a result of application of phase-dependent stimulus pulses. In this case, control device 102 may predict subsequent brain activity based on the baseline brain activity, thereby improving accuracy of a subsequent brain activity prediction relative to predicting with the artifacts included. In some implementations, control device 102 may use a parametric spectral estimation technique to predict brain activity. For example, control device 102 may model band limited oscillations in brain activity using the parametric spectral estimation technique, and may predict subsequent brain activity based on modeling band limited oscillations in brain activity. In some implementations, control device 102 may apply a band-pass optimized autoregressive technique to predict brain activity.

As further shown in FIG. 1, and by reference number 110, control device 102 may provide a stimulus signal to stimulus device 106. For example, control device 102 may cause stimulus device 106 to provide a set of electrical pulses to the brain of the patient during the predicted period of rhythmic brain activity. In this way, control device 102 enables phase-dependent neuromodulation. In some implementations, the set of electrical pulses may be timed in accordance with a phase of brain activity (e.g., within a particular band of oscillatory frequencies), a frequency of brain activity, and/or the like predicted based on past brain activity. In some implementations, control device 102 may cause stimulus device 106 to provide a variable-pulse stimulus. For example, control device 102 may cause electrical pulses to vary in frequency, phase, intensity, and/or the like, thereby enabling reduced power utilization, improved efficacy, and/or the like relative to a constant set of electrical pulses.

As indicated above, FIG. 1 is provided merely as one or more examples. Other examples may differ from what is described with regard to FIG. 1.

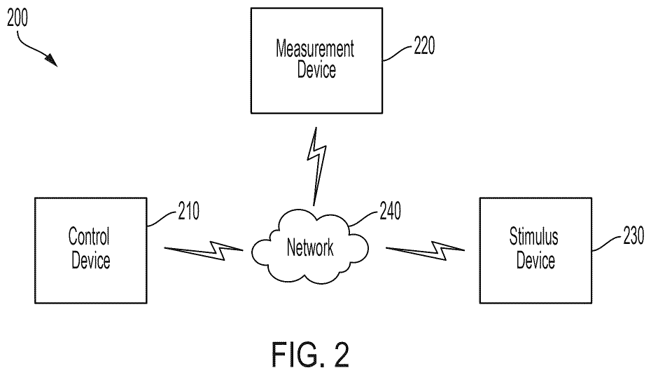

FIG. 2 is a diagram of an example environment 200 in which systems and/or methods described herein may be implemented. As shown in FIG. 2, environment 200 may include a control device 210, a measurement device 220, a stimulus device 230, and a network 240. Devices of environment 200 may interconnect via wired connections, wireless connections, or a combination of wired and wireless connections (e.g., for power transmission, data transmission, and/or the like).

Control device 210 includes one or more devices capable of receiving, generating, storing, processing, and/or providing information associated with phase-dependent neuromodulation. For example, control device 210 may include a communication and/or computing device, such as a computer (e.g., a desktop computer, a laptop computer, a tablet computer, a handheld computer), a medical device, a wearable communication device (e.g., a smart wristwatch, a pair of smart eyeglasses, a wearable medical device, an implantable medical device, etc.), or a similar type of device. In some implementations, control device 210 may be an external device connected to measurement device 220 and/or stimulus device 230. In some implementations, control device 210, measurement device 220, and stimulus device 230 may be an integrated system-on-chip device that is at least partially implanted into a patient.

Measurement device 220 includes one or more devices capable of receiving, generating, storing, processing, and/or providing information associated with a measurement of brain activity. For example, measurement device 220 may include an electrode (e.g., a measurement electrode) for sensing a phase, a frequency, an amplitude, cross-frequency coupling, and/or the like of brain activity of a brain of a patient. In some implementations, measurement device 220 may be a measurement device mounted onto a head of a patient, a measurement device surgically implanted into a head of a patient, and/or the like.

Stimulus device 230 includes one or more devices capable of receiving, generating, storing, processing, and/or providing information associated with stimulation of a brain. For example, stimulus device 230 may include an electrode (e.g., a stimulus electrode) or multiple electrodes for applying an electrical pulse to a brain of a patient. In some implementations, stimulus device 230 may be a stimulation device mounted onto a head of a patient, a stimulation device surgically implanted into a head of a patient, and/or the like.

Network 240 includes one or more wired and/or wireless networks. For example, network 240 may include a cellular network (e.g., a long-term evolution (LTE) network, a code division multiple access (CDMA) network, a 3G network, a 4G network, a 5G network, another type of next generation network, etc.), a public land mobile network (PLMN), a local area network (LAN), a wide area network (WAN), a metropolitan area network (MAN), a telephone network (e.g., the Public Switched Telephone Network (PSTN)), a private network, an ad hoc network, an intranet, the Internet, a fiber optic-based network, a cloud computing network, a BLUETOOTH network using a BLUETOOTH communication protocol, a near-field communication network, or the like, and/or a combination of these or other types of networks. The network can also provide data security, authorization, and/or authentication using one or more public and/or private cryptographic protocols to provide a measure of protected health information assurance.

The number and arrangement of devices and networks shown in FIG. 2 are provided as one or more examples. In practice, there may be additional devices and/or networks, fewer devices and/or networks, different devices and/or networks, or differently arranged devices and/or networks than those shown in FIG. 2. Furthermore, two or more devices shown in FIG. 2 may be implemented within a single device, or a single device shown in FIG. 2 may be implemented as multiple, distributed devices. Additionally, or alternatively, a set of devices (e.g., one or more devices) of environment 200 may perform one or more functions described as being performed by another set of devices of environment 200.

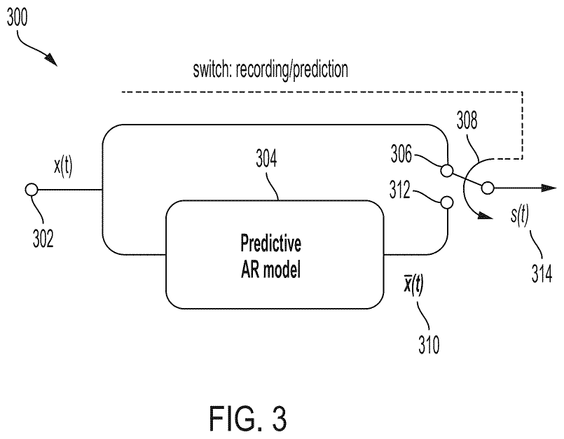

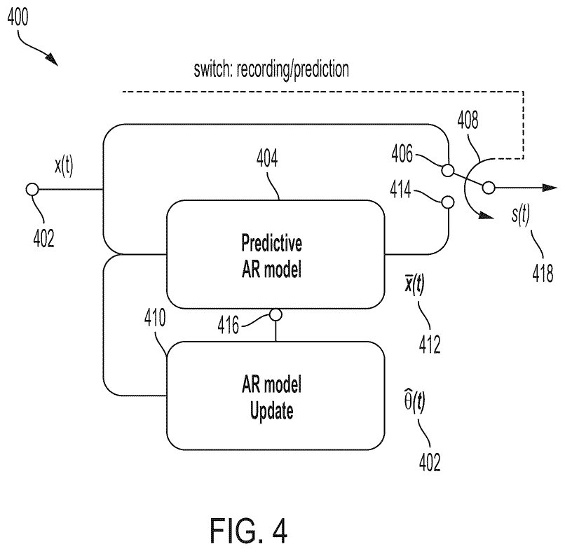

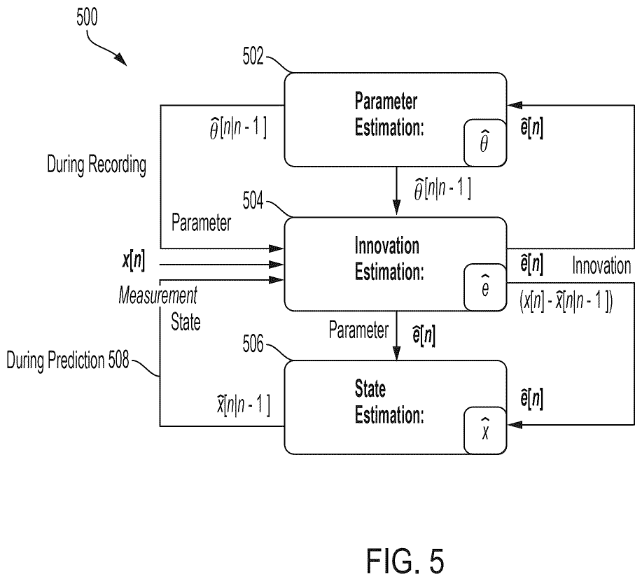

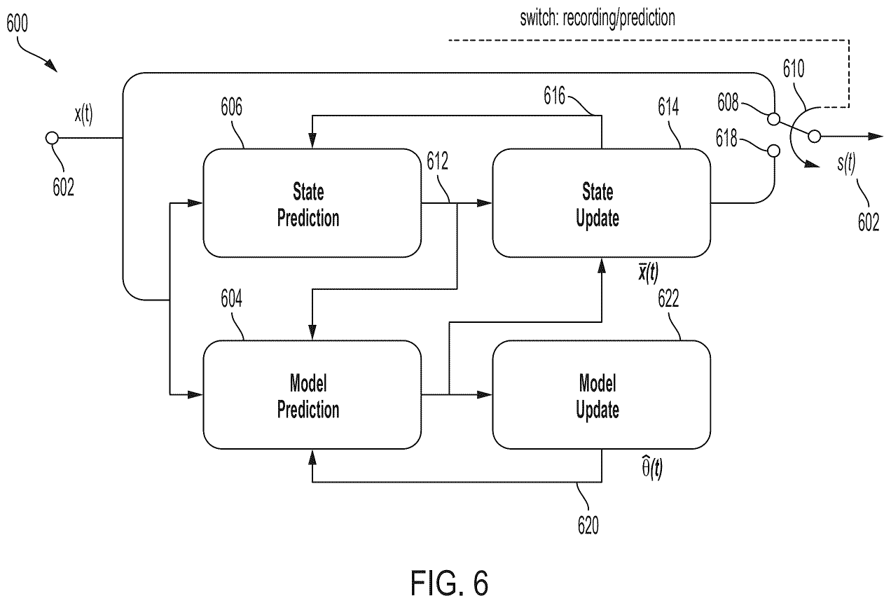

As described below in relation to FIG. 3, FIG. 4, FIG. 5, and FIG. 6, the stimulus artifact is removed by utilizing the output from the predictive model, and by activation of a switching mechanism. The predicted waveform was substituted for the stimulus artifact during active stimulation. The switching time is synchronized with the stimulation trigger, and the stop time is estimated based on the average stereotypic stimulus artifact duration. Additionally, an optimized block is provided for dynamically updating the predictive model parameters.

FIG. 3 shows a first example of a recording/prediction diagram 300 according to examples of the present disclosure. Input signal x(t) 302, which is the first segment 116, is provided to predictive artifact removal (AR) model 304 and provided to first terminal 306 of switch 308. Output 310 from predictive AR model 304, denoted x̅(t), is provided to second terminal 312 of switch 308. Switch 308 outputs signal s(t) 314.

FIG. 4 shows a second example of a recording/prediction diagram 400 according to examples of the present disclosure. Input signal x(t) 402, which is the first segment 116, is provided to predictive AR model 404, provided to first terminal 406 of switch 408, and provided to AR model update (\hat{θ}(t)) 410. Output 412 from predictive AR model 404, denoted x̅(t), is provided to second terminal 414 of switch 408. Output 416 from AR model update 410 is provided to predictive AR model 404. Switch 408 outputs signal s(t) 418.

FIG. 5 shows a third example of a recording/predication diagram 500 according to examples of the present disclosure. Parameter estimation (\hat{θ}) 502 outputs \hat{θ}[n|n−1] and is provided to innovation estimation (ê) 504, which outputs ê[n], which is provided to state estimation (\hat{x}) 506. State estimation (\hat{x}) 506 outputs \hat{x}[n|n−1], which is provided to innovation estimation (ê) 504. Innovation estimation (ê) 504 receives input x[n] 508. Innovation estimation (ê) 504 outputs (x[n]−\hat{x}[n|n−1) to state estimation (\hat{x}) 506.

FIG. 6 shows a fourth example of a recording/prediction diagram 600 according to examples of the present disclosure. Input signal x(t) 602, which is the first segment 116, is provided to predictive AR model 604, provided to state prediction model 606, and provided to first terminal 608 of switch 610. Output 612 from state prediction model 606 is provided to status update 614 and to predictive AR model 604. Output 616 from state update 614 is provided to state prediction 606 and to second terminal 618 of switch 610. Output 620 of model update 622 is provided to predictive AR model 604.

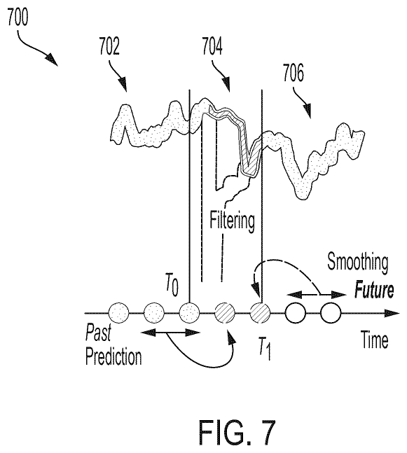

FIG. 7 shows a recorded signal 700 with a modeled predicted signal portion added that has been attributes of prediction, filtering, and smoothing according to examples of the present disclosure. In FIG. 7, a time line is shown representative of the relationships between the filtered (optimizes model parameters), smoothed (compensates the edge effects of artifact removal), and predicted state estimates (remove artifacts). In FIG. 7, it is supposed that we have received measurements at times up to and including t=T₀ and we have stimulation on from t=T₀ to t=T₁. An estimate of the state at t≤T₁ is called the smoothed estimate (for compensating the edge effects). An estimate of the state at t=T₀ is called the filtering (for optimizing the model parameters). An estimate of the state at t≥T₀ is called the prediction (for removing artifacts). Recorded signal 700 is shown segmented into a first portion 702, a second portion 704, and a third portion 706. First portion 702 and third portion 706 represent the recorded brainwave signal from the patient. Second portion 704 represents the modeled, predictive signal that has the stimulus artifact removed. Second portion 704 is modeled to have smooth transitions between first portion 702 and third portion 706 that compensate for edge effects associated with the artifact removal without having any discontinuities in the beginning and end of the modeled signal.

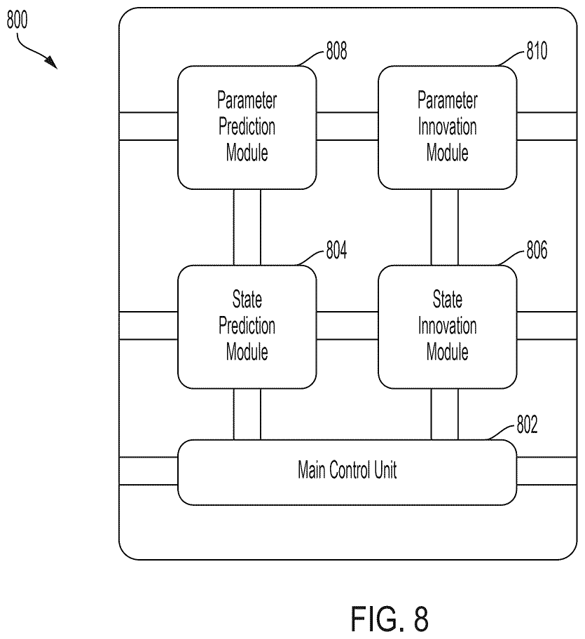

FIG. 8 shows a recording/prediction system diagram 800 according to examples of the present disclosure. Recording/prediction system diagram 800 can comprise main control unit 802, state prediction module 804, state innovation module 806, parameter prediction module 808, and parameter innovation module 810. One or more parametrically adaptive processors are used for nonlinear state-space systems to perform filtering, prediction, and smoothing. It is a combined state/parametric estimator, since it estimates both the states and the model parameters. It is parametrically adaptive, since it adjusts adaptively the model parameters at each time step. One of the advantages of using the combined state/parametric estimator is the possibility of full implementation of the estimator within a field-programmable gate array-based system-on-chip integrated circuits.

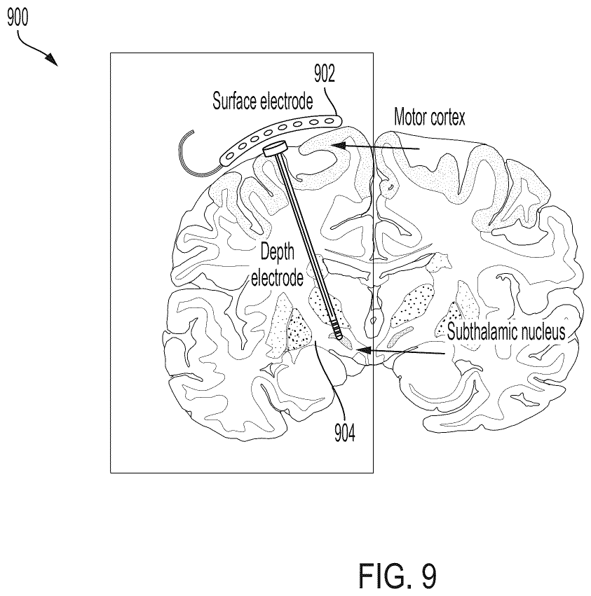

FIG. 9 shows a surface electrode and depth electrode placement 900 according to examples of the present disclosure. Surface electrodes 902 are placed near the motor cortex and depth electrodes 904 are placed near subcortical target structures (including the subthalamic nucleus, globus pallidus interna, and other subcortical targets).

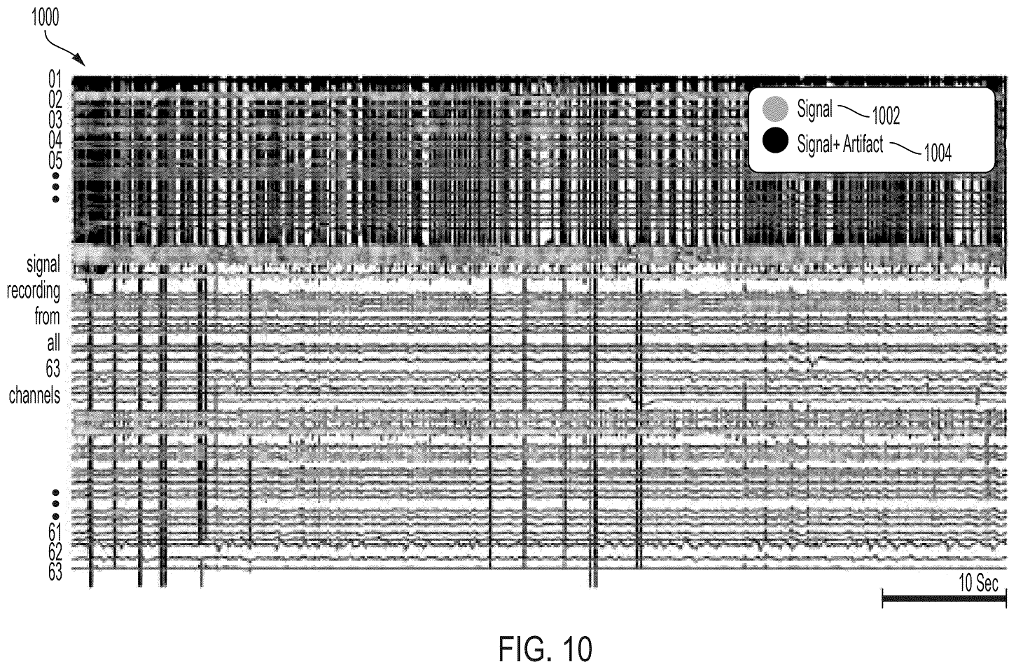

FIG. 10 shows a cortical multichannel recording example during subcortical stimulation 1000 according to examples of the present disclosure: The performance of the artifact removal algorithm 1002 on an estimation of electrophysiological recording 1204 across all contacts in the strip electrode is shown here. The row data are recorded during motor cortex phase-dependent stimulation.

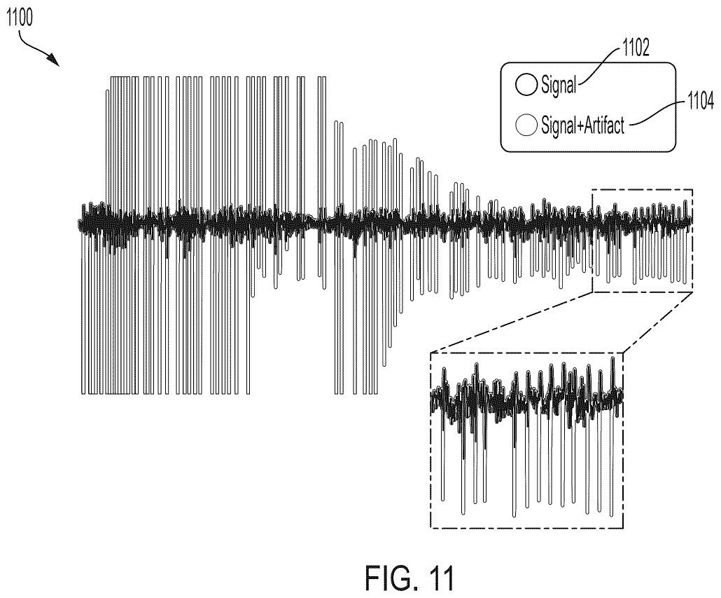

FIG. 11 shows an example of concurrent cortical stimulation and recording 1100 according to examples of the present disclosure: The performance of the artifact removal algorithm 1102 is presented with an estimation of electrophysiological recording 1104 during motor cortex recording and stimulation.

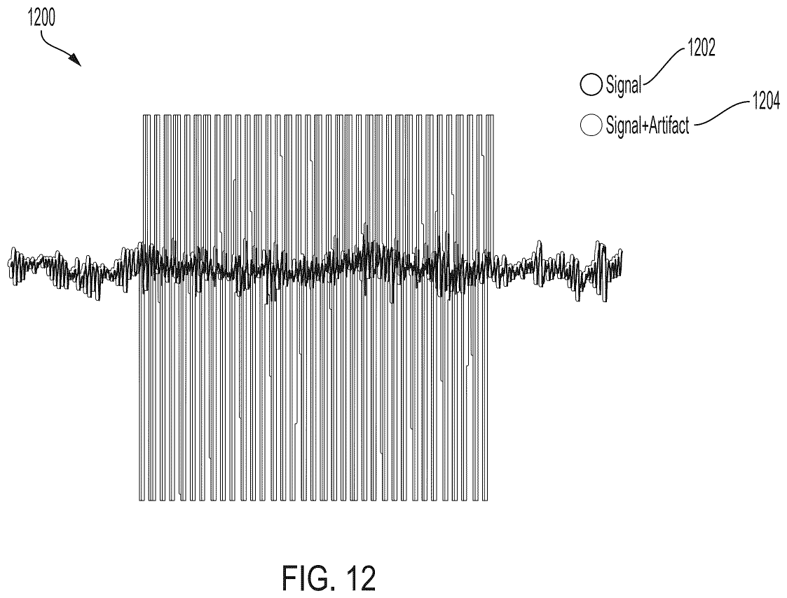

FIG. 12 shows an example of concurrent subcortical stimulation and cortical recording 1200 according to examples of the present disclosure: The performance of the artifact removal algorithm 1202 is presented with an estimation of electrophysiological recording 1204 during subthalamic nucleus stimulation and motor cortex recording.

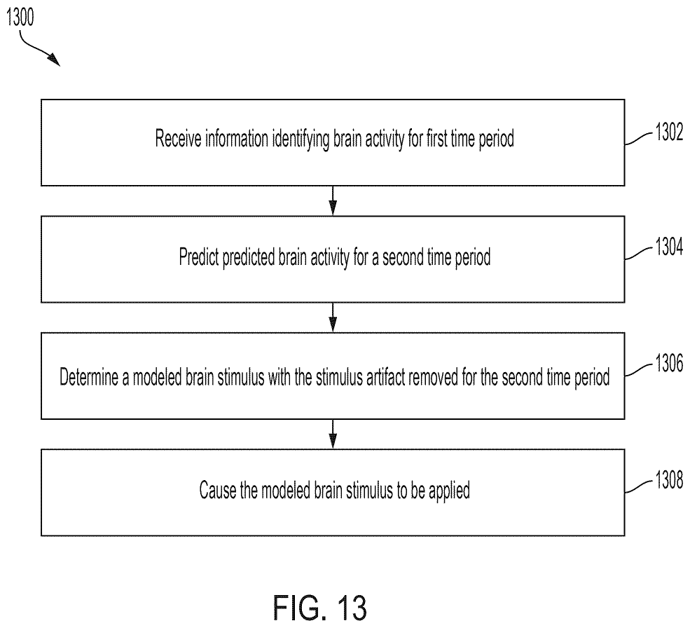

FIG. 13 shows a method for artifact removal for neuromodulation systems 1300 according to examples of the present disclosure. In some implementations, one or more process blocks of FIG. 13 may be performed by a control device (e.g., control device 210). In some implementations, one or more process blocks of FIG. 13 may be performed by another device or a group of devices separate from or including the control device, such as a measurement device (e.g., measurement device 220), a stimulus device (e.g., stimulus device 230), and/or the like.

As shown in FIG. 13, process 1300 may include receiving, from one or more electrodes, information identifying brain activity for a first time period (block 1302). For example, the control device (e.g., using analysis module(s) 1402, processor(s) 1404, storage media 1406, machine learning module(s) 1408, network interface 1407, and/or the like) may receive, from one or more electrodes, information identifying brain activity for a first time period, as described above.

As further shown in FIG. 13, process 1300 may include predicting, based on the information identifying the brain activity for the first time period, predicted brain activity for a second time period that is to occur after the first time period (block 1304). For example, the control device (e.g., using analysis module(s) 1402, processor(s) 1404, storage media 1406, machine learning module(s) 1408, network interface 1407, and/or the like) may predict, based on the information identifying the brain activity for the first time period, predicted brain activity for a second time period that is to occur after the first time period, as described above.

As further shown in FIG. 13, process 1300 may include determining, based on the predicted brain activity for the second time period, a modeled brain stimulus with the stimulus artifact removed for the second time period, wherein the brain stimulus is associated with a frequency and a phase determined based on the predicted brain activity for the second time period (block 1306). For example, the control device (e.g., using analysis module(s) 1402, processor(s) 1404, storage media 1406, machine learning module(s) 1408, network interface 1407, and/or the like) may determine, based on the predicted brain activity for the second time period, a brain stimulus without the stimulus artifact for the second time period, as described above. In some implementations, the modeled brain stimulus is associated with a frequency and a phase determined based on the predicted brain activity for the second time period.

As further shown in FIG. 13, process 1300 may include causing the modeled brain stimulus to be applied in accordance with the frequency and the phase during the second time period (block 1308). For example, the control device (e.g., using analysis module(s) 1402, processor(s) 1404, storage media 1406, machine learning module(s) 1408, network interface 1407, and/or the like) may cause the brain stimulus to be applied in accordance with the frequency and the phase during the second time period, as described above.

Process 1300 may include additional implementations, such as any single implementation or any combination of implementations described below and/or in connection with one or more other processes described elsewhere herein.

In a first implementation, the control device may determine an artifact during the first time period associated with a prior brain stimulus; determine an artifact-removed brain activity for the first time period based on the artifact; and predict the brain activity for the second time period based on the artifact-removed brain activity. In a second implementation, alone or in combination with the first implementation, the phase is a selected phase of a detected brain rhythmic activity, and the brain stimulus includes one or more pulses timed in accordance with the phase. In a third implementation, alone or in combination with one or more of the first and second implementations, the brain stimulus is caused to occur during a period of rhythmic brain activity in accordance with the frequency. In a fourth implementation, alone or in combination with one or more of the first through third implementations, the brain stimulus is a variable-pulse stimulus. In a fifth implementation, alone or in combination with one or more of the first through fourth implementations, the control device may predict the brain activity using a parametric spectral estimation technique for modeling band limited oscillations. In a sixth implementation, alone or in combination with one or more of the first through fifth implementations, the control device may predict the brain activity using a band-pass optimized autoregressive technique. In a seventh implementation, alone or in combination with one or more of the first through sixth implementations, the control device is an external device connected to one or more electrodes disposed onto or into a brain of a patient. In an eighth implementation, alone or in combination with one or more of the first through seventh implementations, the control device is a system-on-chip device at least partially implanted into a patient. In a ninth implementation, alone or in combination with one or more of the first through eighth implementations, process 400 may include determining that the brain activity for the first time period satisfies a threshold and predicting the brain activity for the second time period based at least in part on the brain activity for the first time period satisfying the threshold. In a tenth implementation, alone or in combination with one or more of the first through ninth implementations, the threshold is a beta activity threshold. In an eleventh implementation, alone or in combination with one or more of the first through tenth implementations the threshold is a phase amplitude coupling threshold. In a twelfth implementation, alone or in combination with one or more of the first through eleventh implementations, determining the brain activity includes estimating a phase amplitude coupling in the first time period using a rolling dynamic phase amplitude coupling (PAC) estimation technique. In a thirteenth implementation, alone or in combination with one or more of the first through twelfth implementations the phase amplitude coupling is estimated in a window of less than or equal to 1 second. In a fourteenth implementation, alone or in combination with one or more of the first through thirteenth implementations, the phase amplitude coupling is estimated in a window of less than or equal to 500 milliseconds.

Although FIG. 13 shows example blocks of process 1300, in some implementations, process 1300 may include additional blocks, fewer blocks, different blocks, or differently arranged blocks than those depicted in FIG. 13. Additionally, or alternatively, two or more of the blocks of process 400 may be performed in parallel.

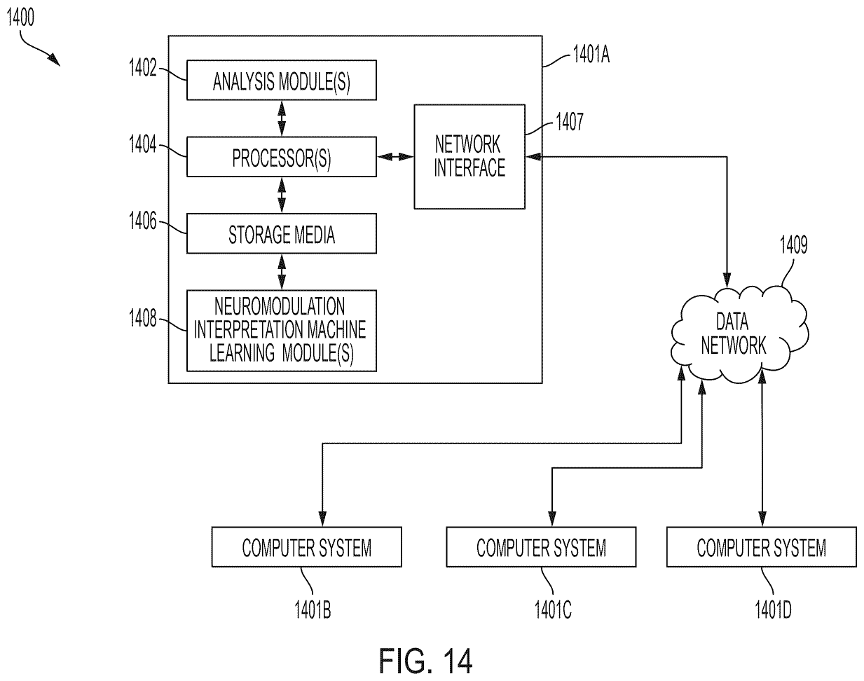

In some embodiments, any of the methods of the present disclosure may be executed by a computing system. FIG. 14 illustrates an example of such a computing system 1400, in accordance with some embodiments. The computing system 1400 may include a computer or computer system 1401A, which may be an individual computer system 1401A or an arrangement of distributed computer systems. The computer system 1401A includes one or more analysis module(s) 1402 configured to perform various tasks according to some embodiments, such as one or more methods disclosed herein. To perform these various tasks, the analysis module 1402 executes independently, or in coordination with, one or more processors 1404, which is (or are) connected to one or more storage media 1406. The processor(s) 1404 is (or are) also connected to a network interface 1407 to allow the computer system 1401A to communicate over a data network 1409 with one or more additional computer systems and/or computing systems, such as 1401B, 1401C, and/or 1401D (note that computer systems 1401B, 1401C and/or 1401D may or may not share the same architecture as computer system 1401A, and may be located in different physical locations, e.g., computer systems 1401A and 1401B may be located in a processing facility, while in communication with one or more computer systems such as 1401C and/or 1401D that are located in one or more data centers, and/or located in varying countries on different continents).

A processor can include a microprocessor, microcontroller, processor module or subsystem, programmable integrated circuit, programmable gate array, or another control or computing device.

The storage media 1406 can be implemented as one or more computer-readable or machine-readable storage media. The storage media 1406 can be connected to or coupled with a neuromodulation interpretation machine learning module(s) 1408. Note that while in the example embodiment of FIG. 14 storage media 1406 is depicted as within computer system 1401A, in some embodiments, storage media 1406 may be distributed within and/or across multiple internal and/or external enclosures of computing system 1401A and/or additional computing systems. Storage media 1406 may include one or more different forms of memory including semiconductor memory devices such as dynamic or static random access memories (DRAMs or SRAMs), erasable and programmable read-only memories (EPROMs), electrically erasable and programmable read-only memories (EEPROMs) and flash memories, magnetic disks such as fixed, floppy and removable disks, other magnetic media including tape, optical media such as compact disks (CDs) or digital video disks (DVDs), BLURAY® disks, or other types of optical storage, or other types of storage devices. Note that the instructions discussed above can be provided on one computer-readable or machine-readable storage medium, or alternatively, can be provided on multiple computer-readable or machine-readable storage media distributed in a large system having possibly plural nodes. Such computer-readable or machine-readable storage medium or media is (are) considered to be part of an article (or article of manufacture). An article or article of manufacture can refer to any manufactured single component or multiple components. The storage medium or media can be located either in the machine running the machine-readable instructions or located at a remote site from which machine-readable instructions can be downloaded over a network for execution.

It should be appreciated that computing system 1400 is only one example of a computing system, and that computing system 700 may have more or fewer components than shown, may combine additional components not depicted in the example embodiment of FIG. 14, and/or computing system 1400 may have a different configuration or arrangement of the components depicted in FIG. 14. The various components shown in FIG. 14 may be implemented in hardware, software, or a combination of both hardware and software, including one or more signal processing and/or application specific integrated circuits.

Further, the steps in the processing methods described herein may be implemented by running one or more functional modules in an information processing apparatus such as general purpose processors or application specific chips, such as ASICs, FPGAs, PLDs, or other appropriate devices. These modules, combinations of these modules, and/or their combination with general hardware are all included within the scope of protection of the invention.

Neuromodulation and/or artifact removal interpretations, models and/or other interpretation aids may be refined in an iterative fashion; this concept is applicable to embodiments of the present methods discussed herein. This can include use of feedback loops executed on an algorithmic basis, such as at a computing device (e.g., computing system 1400, FIG. 14), and/or through manual control by a user who may make determinations regarding whether a given step, action, template, model, or set of curves has become sufficiently accurate for the evaluation of the signal(s) under consideration.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. Moreover, the order in which the elements of the methods are illustrated and described may be re-arranged, and/or two or more elements may occur simultaneously. The embodiments were chosen and described in order to best explain the principles of the invention and its practical applications, to thereby enable others skilled in the art to best utilize the invention and various embodiments with various modifications as are suited to the particular use contemplated.

What is claimed is:

1. A method, comprising:
receiving, from one or more electrodes, information identifying brain activity with a stimulus artifact signal for a first time period;
predicting, based on the information identifying the brain activity for the first time period, predicted brain activity without the stimulus artifact signal for a second time period that is to occur after the first time period;
inserting the predicted brain activity into the information for a second time period;
determining, based on the predicted brain activity for the second time period, a brain stimulus for the second time period; and
causing the brain stimulus to be applied the second time period.

2. The method of claim 1, wherein the brain stimulus is associated with a frequency and a phase determined based on the predicted brain activity for the second time period.

3. The method of claim 1, further comprising:
determining the stimulus artifact signal during the first time period associated with a prior brain stimulus; and
determining an artifact-removed brain activity for the first time period based on the artifact.

4. The method of claim 3, wherein predicting the brain activity for the second time period further comprises:
predicting the brain activity for the second time period based on the artifact-removed brain activity.

5. The method of claim 1, wherein the brain stimulus is caused to occur during a period of rhythmic brain activity.

6. The method of claim 1, wherein predicting the brain activity for the second time period further comprises:
predicting the brain activity using a parametric spectral estimation technique for modeling band limited oscillations.

7. A device, comprising:
one or more memories; and
one or more processors communicatively coupled to the one or more memories, configured to:
receiving, from one or more electrodes, information identifying brain activity with a stimulus artifact signal for a first time period;
predicting, based on the information identifying the brain activity for the first time period, predicted brain activity without the stimulus artifact signal for a second time period that is to occur after the first time period;
inserting the predicted brain activity into the information for a second time period;
determining, based on the predicted brain activity for the second time period, a brain stimulus for the second time period; and
causing the brain stimulus to be applied the second time period.

8. The device of claim 7, wherein the brain stimulus is associated with a frequency and a phase determined based on the predicted brain activity for the second time period.

9. The device of claim 7, wherein the one or more processors communicatively coupled to the one or more memories are further configured to:
determining the stimulus artifact signal during the first time period associated with a prior brain stimulus; and
determining an artifact-removed brain activity for the first time period based on the artifact.

10. The device of claim 9, wherein predicting the brain activity for the second time period further comprises:
predicting the brain activity for the second time period based on the artifact-removed brain activity.

11. The device of claim 7, wherein the brain stimulus is caused to occur during a period of rhythmic brain activity.

12. The device of claim 7, wherein predicting the brain activity for the second time period further comprises:
predict the brain activity using a parametric spectral estimation technique for modeling band limited oscillations.

13. A non-transitory computer readable medium comprising instructions that when executed by a hardware processor cause the hardware processor perform a method, comprising:
receiving, from one or more electrodes, information identifying brain activity with a stimulus artifact signal for a first time period;
predicting, based on the information identifying the brain activity for the first time period, predicted brain activity without the stimulus artifact signal for a second time period that is to occur after the first time period;
inserting the predicted brain activity into the information for a second time period;
determining, based on the predicted brain activity for the second time period, a brain stimulus for the second time period; and
causing the brain stimulus to be applied the second time period.

14. The non-transitory computer readable medium of claim 13, wherein the brain stimulus is associated with a frequency and a phase determined based on the predicted brain activity for the second time period.

15. The non-transitory computer readable medium of claim 13, further comprising:
determining the stimulus artifact signal during the first time period associated with a prior brain stimulus; and
determining an artifact-removed brain activity for the first time period based on the artifact.

16. The non-transitory computer readable medium of claim 15, wherein predicting the brain activity for the second time period further comprises:
predicting the brain activity for the second time period based on the artifact-removed brain activity.

17. The non-transitory computer readable medium of claim 16, wherein the brain stimulus is caused to occur during a period of rhythmic brain activity.

18. The non-transitory computer readable medium of claim 13, wherein predicting the brain activity for the second time period further comprises:
predicting the brain activity using a parametric spectral estimation technique for modeling band limited oscillations.
